(19) United States
(12) Patent Application Publication
(10) Pub. No.: US 2010/0113469 A1
(43) Pub. Date: May 6, 2010

(54) COMBINATION THERAPY FOR THE TREATMENT-OF LOWER URINARY TRACT SYMPTOMS

(75) Inventors: Tara Frenkl, Blue Bell, PA (US); Stuart A. Green, East Brunswick, NJ (US); Enan Macintyre, Scotch Plains, NJ (US); Sander G. Mills, Scotch Plains, NJ (US)

Correspondence Address: MERCK, P O BOX 2000, RAHWAY, NJ, 07065-0907, US

(73) Assignee: MERCK & CO., INC., Rahway, NJ (US)

(21) Appl. No.: 12/532,731

(22) PCT Filed: Mar. 25, 2008
(86) PCT No.: PCT/US2008/003873

§ 371 Date: Sep. 23, 2009

Related U.S. Application Data
(60) Provisional application No. 60/920,755, filed on Mar. 29, 2007.

(51) Int. Cl. A61K 31/4439; A61K 31/58; A61K 31/439; A61K 31/445; A61K 31/403; A61K 31/454; A61K 31/46; A61P 13/00

(52) U.S. Cl. 514/252.17; 514/284; 514/305; 514/327; 514/412; 514/266.24; 514/323; 514/278; 514/340; 514/342

(57) ABSTRACT

This invention concerns compositions for the treatment of Lower Urinary Tract Symptoms (LUTS), and especially LUTS which results from benign prostatic hypertrophy. The compositions of the invention comprise a Beta-3 agonist described below, optionally in combination with a 5-alpha reductase inhibitor, or an NK-1 antagonist or an alpha-1 adrenergic antagonist or an anti-muscarinic agent. The invention also includes compositions comprising a beta-3 agonist and two additional active agents selected from a 5-alpha reductase inhibitor, an NK-1 antagonist, an alpha-1 adrenergic antagonist or an anti-muscarinic agent.

### FIELD OF THE INVENTION

This invention concerns compositions for the treatment of Lower Urinary Tract Symptoms (LUTS), and especially LUTS which results from benign prostatic hypertrophy. The compositions of the invention comprise a Beta-3 agonist (Beta-3 adrenergic receptor agonist) described below, optionally in combination with a 5-alpha reductase inhibitor, or an NK-1 antagonist or an alpha-1 adrenergic antagonist or an anti-muscarinic agent. The invention also includes compositions comprising a beta-3 agonist and two additional active agents selected from a 5-alpha reductase inhibitor, an NK-1 antagonist and an alpha-1 adrenergic antagonist.

### BACKGROUND OF THE INVENTION

BPH is a progressive, nearly universal condition in aging men characterized by a nodular enlargement of prostatic tissue resulting, through obstruction of the urethra, in variable degrees of bladder outlet obstruction. The disorder is not a major cause of death, but it is a leading cause of morbidity in elderly men, significantly affecting quality of life. BPH is associated with a variety of lower urinary tract symptoms. Chronic consequences of BPH can include hypertrophy of the bladder smooth muscle, urinary retention, bladder stones, an increased incidence of urinary tract infection, incontinence, and renal failure. The specific biochemical, histological and pharmacological properties of the prostate adenoma leading to the bladder outlet obstruction are not yet known. However, the development of BPH is considered to be an inescapable phenomenon for the aging male population. BPH is commonly seen in men over the age of 50, is observed in approximately 70% of males over the age of 70, and becomes nearly universal with advancing age with 90% incidence at the age of 80 years [Berry et al, J. Urol., 132:474 479, 1984].

Lower urinary tract symptoms (LUTS) in men include, but are not, restricted to a complex of obstructive (voiding) and irritative (storage or filling) symptoms, which include increased frequency, nocturia, poor urinary stream and hesitancy or delay in starting urinary flow. LUTS are recognized as arising from changes in urethral resistance induced by the enlarging prostate as well as contraction of the prostatic smooth muscle. The resulting increase in urethral resistance restricts the outflow of urine and causes secondary changes are induced in the bladder. A characteristic pattern of unstable bladder contractions, also known as irritable bladder, is often observed in men with morphological BPH.

Though the exact etiology of origin of these symptoms is not distinctly clear, two components, a static component and a dynamic component, contribute to obstruction. Prostatic enlargement or hyperplasia of prostate gland physically impinges on the free flow of fluids through the male urethra and leads to varying degrees of bladder obstruction. This component has been referred as the static component [Caine M, J. Urol., 136:1 4, 1986]. Histologically, BPH is characterized by glandular (epithelial) and stromal (fibromuscular) hyperplasia. The observed increase in cell number may be due to epithelial and stromal proliferation or to impaired programmed cell death leading to cellular accumulation. During early BPH development, the disease may be predominantly characterized by an increased number of nodules, however the subsequent growth is generally slow (McNeal, 1990). In a second phase of evolution, there is a significant increase in large nodules. In the first phase, the glandular nodules tend to be larger than the stromal nodules. In the second phase, when the size of individual nodules is increasing, the size of glandular nodules clearly predominates.

The dynamic component of obstruction is secondary to increased adrenergic innervation of the prostatic and urethral smooth muscle, resulting in increased urethral resistance. The irritative symptoms have been closely associated with bladder dysfunction, which was believed to be a consequence of bladder outlet obstruction [Anderson K E, Brit. J. Urol., 85 Suppl: 12-18, 2000].

Standard treatments for BPH involve surgical or pharmacological intervention. Surgical intervention involves removal of the prostate adenoma via open “simple” prostectomy,endoscopic transurethral resection, and “minimally invasive” office procedures Surgery is generally recommended when the patient has severe symptoms or the sequela of BPH noted above (recurrent UTI, recurrent gross hematuria, bladder stones, renal insufficiency, or large bladder diverticula) (McConnell et al, 1994; Denis et al, 1998). These surgical interventions are limited by their associated significant morbidities or limited efficacy resulting in the persistence and recurrence of obstructive and irritative symptoms. Therfore, pharmacologic rather than surgical intervention is recommended for patients exhibiting mild to moderate symptoms.

Presently, pharmacological interventions in the treatment of BPH can be categorized into two main categories: alpha-1 adrenergic receptor antagonists and 5-alpha reductase inhibitors.

The development and enlargement of the prostate gland is dependent on the potent androgen, 5-alpha-dihydrotesterone (DTH). 5-alpha-reductase converts testosterone to DHT in the prostate gland, liver and skin. DTH induces androgenic effects by binding to androgen receptors in the cell nuclei of these organs. Finasteride and dutasteride are competitive inhibitors of and therefore block the conversion of testosterone to DHT. Finasteride is a selective for type 2 5α-reductase whereas and dutasteride inhibits both type 1 and type 2 5α-reductase inhibitor Both finasteride and dutasteride produce a rapid reduction in serum DTH concentration which eventually leads to a reduction in prostate size. [Wilde et al, Drugs, 57: 557-581, 1999; Roehrborn et al: Urology 2002;60:434-441.] Maximal symptom improvement may take 6-12 months after treatment has begun and requires continuous therapy thereafter. [Gormley et al. N Engl J Med 1992;327:1185-1191, Roehrborn et al: Urology 2004;63:709-715.]. These medications do not appear to affect the smooth muscle of the prostate or dynamic component of bladder outlet obstruction.

See more recently, Inhibitors of 5alpha-reductase in the treatment of benign prostatic hyperplasia T H Tarter and E D Vaughan, Jr. Curr Pharm Des. 2006;12(7):775-83

The second class of compounds, known as alpha-1 adrenergic receptor antagonists, is available to treat BPH and are believed to address the dynamic component of symptomatic BPH. Alpha adrenoceptors are members of a larger G protein-coupled adrenergic receptors family, which mediate the actions of the endogenous catecholamines, norepinephrine and epinephrine, resulting in smooth muscle contraction. cDNA's encoding three distinct alpha-1 adrenoceptor subtype (alpha-1a, alpha-1b and alpha-1d) and three distinct alpha-2 adrenoceptor subtypes (alpha-2a, alpha-2b and alpha-2c) have been cloned, expressed stably in cells and resultant protein characterized pharmacologically, [Schwinn et al, J. Pharmacol. Expel: Ther., 272: 134-142, 1995, Hieble et al Pharmacol. Rev., 47:267-70, 1995].

Human lower urinary tract contains both alpha-1 and alpha-2 adrenoceptors. Alpha-1 adrenoceptors predominate in prostate and bladder trigone, [Price et al J. Urol., 150:546-551, 1993; Goopel et al, Urol. Res., 25:199-206, 1997], and have been shown to be functionally important in mediating smooth muscle contraction [Forray et al, Mol. Pharmacol., 45:703-708, 1994, Lepor et al J. Pharmacol. Exper. Ther., 270:722-727, 1994; Hiebleet al, Eur. Pharmacol., 107:111-117, 1985, Chappel et al, Br. J. Urol., 63.487-496, 1989].

Alpha-1 adrenoceptor antagonists relax prostatic-urethral smooth muscle by blocking the alpha-1 mediated effects on endogenous tone and thus cause relaxation of the prostate smooth muscle resulting in a decrease in urethral resistance and increased uroflow. Currently available alpha-1 adrenoceptor antagonists, such as prazosin, terazosin, doxazosin and alfuzosin have been found to relieve both the obstructive and the irritative bladder symptoms associated with BPH [Chapple, Brit J. Urol., 1:47-55, 1995, Kawabe and Niijima, Urol. 42:280-284, 1987, Lepor et al, J. Urol., 148:1467 1474, 1992, Reuther and Aagard, Urol. 39:312-313, 1984, Serels and Stein, Neurourol. Urodyn., 17:31-36, 1998]. Patients with mild to moderate BPH experience a moderate improvement in symptoms. The magnitude of the effect is considerably less than that achieved after surgery. Common side effects of alpha blockers include as postural hypotension, dizziness, syncope and retrograde ejaculation.

See recent articles—Lower urinary tract symptoms suggestive of benign prostatic hyperplasia: latest update on alpha-adrenoceptor antagonists S. Milani and B. Djavan British Journal of Urology International June 2005;95 Suppl 4:29-36; Alfuzosin: an alpha1-receptor blocker for the treatment of lower urinary tract symptoms associated with benign prostatic hyperplasia M M Elhilali Expert Opin Pharmacother. April 2006;7(5):583-96; See also Thomas C. Westfall and David P. Westfall, Adrenergic Agonists and Antagonists, chapter 10, in L. L. Brunton, J. S. Lazo and K. L. Parker, Goodman & Gilman's The Pharmaceutical Basis of Therapeutics, 11th edition, 2006; McGraw-Hill: New York.

A need for novel therapies for BPH and LUTS definitely exists. New classes of compounds which show potential for LUTS and overactive bladder in both men and women include β3 adrenergic receptors (βAR) agonist and Neurokinin 1 receptor antagonists.

β3AR are the most prevalent βAR subtype expressed on human detrusor smooth muscle. See Takeda H, Yamazaki Y, Akahane M, Akahane S, Miyata H, Igawa Y, Nishizawa O. Characterization of β-Adrenoceptor Subtype in Bladder Smooth Muscle in Cynomolgus Monkey, Jap J. Pharmacol 2002;88:108-13. Like other βAR subtypes (i.e., β1AR, β2AR), agonist-promoted stimulation of membrane-bound β3AR results in increased intracellular levels of cyclic adenosine monophosphate (cAMP) via activation of G proteins and adenylyl cyclase. In isolated human bladder smooth muscle, activation of β3AR using subtype-selective agonists results in smooth muscle relaxation. Anticholinergics, which are the current mainstay of treatment for urinary frequency, urinary urgency and incontinence, also cause smooth muscle relaxation via inhibition of acetylcholine-promoted smooth muscle contraction. Thus, it is reasonable to hypothesize that other agents that relax bladder smooth muscle, such as β3AR agonists, may be effective for treating urinary urgency.

β2AR are also expressed on human detrusor, and clenbuterol, a β2AR-selective agonist, has been approved for the treatment of urinary frequency, urinary urgency in Japan. However, β2AR agonists are associated with significant mechanism-based side effects such as tachycardia due to stimulation of cardiac β2AR. Thus, use of β3AR-selective agonists may offer a therapeutic advantage by promoting selective detrusor relaxation while minimizing significant mechanism-based side effects such as those associated with anticholinergics or β2AR agonists.

The presence of β3 adrenergic receptors (β3AR) in detrusor smooth muscle of various species, including human, rat, guinea pig, rabbit, ferret, dog, cat, pig and non-human primate has been evaluated using radioligand binding and/or functional studies in vitro. The latter typically involve measurement of relaxation in strips of bladder tissue pre-contracted using muscarinic agonists, endothelin agonists or KCl. Both approaches are complicated by the species differences among β3AR which impact the potency and pharmacological specificity of putative agonists and antagonists used to characterize β3AR. Nevertheless, in aggregate such pharmacological studies indicate there are marked species differences in the receptor subtypes mediating relaxation of the isolated detrusor, where β1AR predominate in cats and guinea pig, β2AR predominate in rabbit, and β3AR contribute or predominate in dog, rat, ferret, pig, cynomolgus and human detrusors. Expression of β3AR subtypes in the human and rat detrusor has been examined by a variety of techniques, and the presence of β3AR was confirmed using in situ hybridization and/or reverse transcription-polymerase chain reaction (RT-PCR). Real time quantitative PCR analyses of β1AR, β2AR and β3AR mRNAs in bladder tissue from patients undergoing radical cystectomy revealed a preponderance of β3AR mRNA (97% cf 1.5% for β1AR mRNA and 1.4% for β2AR mRNA). Moreover, β3AR mRNA expression was equivalent in control and obstructed human bladders, as was relaxation evoked by the human β3AR agonist L-755507 in vitro. These data suggest that bladder outlet obstruction does not result in downregulation of β3AR, or in alteration of β3AR-mediated detrusor relaxation. β3AR responsiveness also has been compared in bladder strips obtained during cystectomy or enterocystoplasty from patients judged to have normal bladder function, and from patients with detrusor hyporeflexia or hyperreflexia. No differences in the extent or potency of β3AR agonist mediated relaxation were observed, consistent with the concept that the β3AR activation is an effective way of relaxing the detrusor in normal and pathogenic states.

Functional evidence in support of an important role for the β3AR in urine storage emanates from studies in vivo. Following intravenous administration to rats, the rodent selective β3AR agonist CL316243 reduces bladder pressure and in cystomeric studies increases bladder capacity leading to prolongation of micturition interval without increasing residual urine volume. In experimental models in rats detrusor instability can be evoked by outlet obstruction, with consequent bladder hypertrophy and spontaneous bladder contractions. Bladder hyperreflexia can be evoked by intravesicular instillation of acetic acid, PGE2 or other stimuli which activate sensory afferent fibers with attendant reduced voiding interval and spontaneous bladder contractions during filling. Hyperreflexia may also be induced by cerebral infarction (middle cerebral artery occlusion), the effects of which are attributed to decreased inhibitory suprapontine control. In the hyperreflexia paradigms, CL316243 administered intravenously dose-dependently normalizes voiding interval and produces decreases in voiding amplitude and increases in bladder capacity and compliance. In the detrusor instability paradigm CL316243 administered orally results in dose-dependent inhibition of spontaneous bladder contractions. See Takeda H, Yamazaki Y, Akahane M, Igawa Y, Ajisawa Y, Nishizawa O. Role of the β3-Adrenoceptor in Urine Storage in the Rat: Comparison Between the Selective β3-Adrenoceptor Agonist, CL316,243, and Various Smooth Muscle Relaxants. J Pharm Exp Ther 2000;293:939-45. See Woods M, Carson N, Norton N, Wesley S, Jeffery H, Argentieri T M. Efficacy of the [Beta]3-Adrenergic Receptor Agonist CL-316243 on Experimental Bladder Hyperreflexia and Detrusor Instability in the Rat. J Urol 2001;166:1142-7. See Takeda H, Yamazaki Y, Igawa Y, Kaidoh K, Akahane S, Miyata H, Nishizawa O, Akahane M, Andersson K E. Effects of β3-Adrenoceptor Stimulation on Prostaglandin E2-Induced Bladder Hyperactivity and on the Cardiovascular System in Conscious Rats. Neurology and Urodynamics 2002;21:558-65. Kaidoh K, Igawa Y, Takeda H, Yamazaki Y, Akahane S, Miyata H, Ajisawa Y, Nishizawa O, Andersson K E. Effects of Selective [beta]2 and [beta]3-Adrenoceptor Agonists on Detrusor Hyperreflexia in Conscious Cerebral Infarcted Rats. J Urol 2002;168:1247-52.

The second novel class of compounds which may have utility in overactive bladder and LUTS are Neurokinin 1 antagonists which may modulate bladder sensory input to the central nervous system. Adequate sensory input is a prerequisite for normal bladder control and changes in sensory mechanisms may give rise to disturbances in bladder function. Thus, it has been proposed that urge incontinence is “a disease of bladder sensors”. See Klein, L. A.: J Urol., 139: 1010-10-14, 1998. In spinal health, afferent activity from the bladder is mediated largely by the myelinated Aδ-fibers that pass through the spinal tracts to the brainstem and then to the pontine micturition center. After spinal disruption, a different type of afferent pathway emerges that is mediated by unmyelinated C-fibers that are sensitive to capsaicin. It is thought that these primary afferent C-fibers drive the spinal segmental reflex pathway and may be involved in pathological conditions of the bladder including overactivity and incontinence.

A renewed interest in tachykinins (TK) and especially NK receptor antagonists, on the micturition reflex is due to the recent introduction of C-fiber neurotoxins (capsaicin and resinferatoxin) in urology for the treatment of both idiopathic micturition disorders and those related to neurological dysfunctions such as multiple sclerosis, Parkinson's disease and spinal cord injuries. See Maggi, C. A., Barbanti, G., Santicioli, P., Beneforti, P., Misuri, D., Meli, A. and Turini, D.: Cystometric evidence that capsaicin-sensitive nerves modulate the afferent branch of micturition reflex of humans. J. Urol., 142: 150, 1989. Lazzeri, M., Beneforti, P., Spinelli, M., Barbagli G., Turini D. Intravesical resiniferatoxin for the treatment of hypersensitive disorders: a randomized placebo controlled study. J Urol., 164:676-679, 2000. Dasgupta, P. and Fowler, C. J. Chilies from antiquity to urology. Brit. J. Urol., 80:845, 1997. Lecci A., birder, L., Meini, S., Giuliani, S., Tramontana, M., Criscuoli, M. Capsaicin and the micturition reflex: actions of tachykinins and other transmitters. Curr. Top. Pharmacol., 4; 193-220, 1998. M. B. Chancellor and W. C. de Groat, Intravesical capsaicin and resiniferatoxin therapy; spicing up the ways to treat the overactive bladder. J. Urol. 162; 3-11, 1999. Capsaicin, instilled directly into the bladder, was the first such agent used and it has been reported to achieve beneficial effects (i.e. increased bladder capacity) in ˜70% of patients. Resinferatoxin, which is ˜100-fold more potent than capsaicin, causes prolonged inactivation of C-fibers without the initial stimulatory effects. See Avelino, A., Cruz, F., Coimbra, A. Intravesical resinferatoxin desensitizes rat bladder sensory fibers without causing intense noxious excitation. A C-fos study. Eur J Pharmacol., 378; 17-25, 1999. The introduction of these agents into humans was supported by several animal studies that showed local or systemic treatment with capsaicin or resiniferatoxin, at doses that depleted substance P and neurokinin A in the bladder, caused an increase in bladder capacity and reduced bladder hyperactivity. See Holzer-Petsche, U. and Lembeck, F. Systemic capsaicin treatment impairs the micturition reflex in the rat. Br. J. Pharmacol. 83; 935-941, 1984. Cheng, C. I., Ma, C. P. and de Groat, W. C. Effect of capsaicin on micturition and associated reflexes in rats. Amer. J. Physiol., part 2,34; R132, 1993. Cheng, C. I., Ma, C. P. and de Groat, W. Effect of capsaicin on micturition and associated reflexes in chronic spinal rats. Brain Res., 678; 40-48, 1995. Maggi, C. A., Santicioli, P. and Meli, A.: The effects of topical capsaicin on rat urinary bladder motility in vivo. Eur. J. Pharmacol., 103;41-51, 1984. Santicioli, P., Maggi, C. A. and Meli, A.: The effect capsaicin pretreatment on the cystometrograms of urethrane anesthetized rats. J. Urol., 133; 700-708, 1985. Thus, a possible role of tachykinins as sensory transmitters in the micturition reflex has been postulated and NK₁ and/or NK₂ receptor antagonists may induce the same effects as capsaicin by inhibiting the sensorial input from the bladder to the spinal cord, thus increasing the threshold to initiate micturition.

The effects of selective NK₁ and NK₂ receptor antagonists have been studied in various animal models of bladder function. Using a cyclophosphamide-induced model of bladder overactivity, it has been shown that two NK₁ antagonists (GR 82334 and RP 67580) increased the volume threshold after i.t., but not i.v. administration. A moderate response in this model was also observed with the NK₂ antagonist SR 48968 (10 nmol/rat), however the i.v. co-administration of NK₁ and NK₂, antagonists did not modify urodynamic variables in either vehicle- or cyclophosphamide-treated rats. See Lecci, A., Giuliani, S., Santicioli, P., Maggi, C. A. Involvement of spinal tachykinin NK1 and NK2 receptors in detrusor hyperreflexia during chemical cystitis in anaesthetized rats. Eur J. Pharmacol., 259; 129-135, 1994. Using RP 67580 and SR 48968, Ishizuka et al., found that spinal NK₁ receptors are involved in the micturition reflex induced by bladder filling in animals with bladder hypertrophy secondary to outflow obstruction. See Ishizuka, O., Igwana, Y., Lecci, A., et al. 1994. Role of intrathecal tachykinin for micturition in unanesthetized rats with and without bladder outlet obstruction. Br. J. Pharmacol. 113, 111-123. Another study determined that intrathecal administration of GR 82334 blocked capsaicin-induced micturition reflex in rats. Importantly, at the same doses proved effective in the chemonociceptive reflex, GR 82334 did not affect the micturition reflex induced by bladder filling or the force of contraction induced by perineal pinching. See Lecci, A., Giuliani, S., Maggi, C. A. Effect of the NK-1 receptor antagonist GR 82334 on reflexly-induced bladder contractions. Life Sciences, 51; 277-280, 1992.

Scientists at Takeda Laboratories have investigated the effects of TAK-637 on lower urinary tract function in guinea pigs and cats. Kamo and Doi, reported that in decerebrate cats, TAK-637 (0.1, 0.3, 1 and 3 mg/kg i.v.) produced a dose-dependent increase in bladder capacity (maximal increase was 94%) without any significant reduction in voiding efficiency. TAK-637 at 3 mg/kg i.v. did not inhibit the micturition reflex induced by electrical stimulation of the rostral brainstem near the locus coeruleus, indicating that it does not impair the efferent pathways of the micturition reflex. These results suggest that TAK-637 increases bladder storage capability without inhibiting the voiding function of the lower urinary tract, presumably by inhibiting the afferent pathway of the micturition reflex rather than the efferent pathway. The systemic administration of TAK-637 decreased the number but not the amplitude of distension-induced rhythmic bladder contractions in guinea pig, an effect which was also observed in animals with severed spinal cords. TAK-637 also inhibited the micturition reflex induced by topical application of capsaicin (which stimulates primary afferent nerve endings in the bladder wall) onto the surface of the bladder dome. These results suggest that TAK-637 inhibits sensory transmission from the bladder evoked by both physiological and nociceptive stimuli by blocking tachykinin NK₁ receptors, almost certainly at the level of the spinal cord. Furthermore, TAK-637 inhibits the spinal vesico-vesical reflex induced by electrical stimulation of the proximal cut end of the pelvic nerve in spinal animals, but not bladder contractions induced by electrical stimulation of the distal cut end of the nerve. Tissue bath studies showed that TAK-637 had no effect on carbachol or electrical field stimulation induced contractions of isolated bladder strips, whereas other drugs used for abnormally frequent micturition inhibited both contractions. These results suggest that TAK-637 inhibits the micturition reflex by acting, at least in part, on NK₁ receptors in the spinal cord, a mechanism of action clearly different from antimuscarinics or spasmolytics

NK-1 receptor antagonists, and in particular, those whose use is claimed herein, are also believed to be useful in the treatment of Lower Urinary Tract Symptoms (LUTS).

See Moller, et. al., BMJ 2000; 320: 1429-1432 (27 May); Pinnock and Marshall, MJA 1997; 167: 72-75 (21 July); Moller, et. al., Obstetrics & Gyneology 2000; 96:446-451; and Clinical Practice Guidelines: The Management of Uncomplicated Lower Urinary Tract Symptoms in Men, UHMRC 2000.

See more recently—Efficacy and safety of a neurokinin-1 receptor antagonist in postmenopausal women with overactive bladder with urge urinary incontinence S A Green, A Alon, J. Ianus, K S McNaughton, C A Tozzi, T F Reiss J Urol. December 2006;176(6 Pt 1):2535-40 and above referenced filings

Additional references include WO2005/092342, published Oct. 6, 2005 and US2005/0101607, published May 12, 2005.

### SUMMARY OF THE INVENTION

This invention concerns compositions for the treatment of Lower Urinary Tract Symptoms (LUTS), and especially LUTS which results from benign prostatic hypertrophy. The compositions of the invention comprise a Beta-3 agonist described below, optionally in combination with a 5-alpha reductase inhibitor, or an NK-1 antagonist or an alpha-1 adrenergic antagonist. The invention also includes compositions comprising a beta-3 agonist of Formula (I) and two additional active agents selected from a 5-alpha reductase inhibitor, an NK-1 antagonist and an alpha-1 adrenergic antagonist.

### DETAILED DESCRIPTION OF THE INVENTION

In one aspect the invention is directed to a pharmaceutical composition for the treatment of lower urinary tract symptoms (LUTS), especially LUTS which results from benign prostatic hypertrophy (BPH), comprising a beta 3 agonist selected from
N-[4-[2-[[2-hydroxy-2-(pyridin-3-yl)ethyl]amino]ethyl]phenyl]-4-[4-(3-cyclopentylpropyl)-5-tetrazolon-1-yl]benzenesulfonamide; and
2N-[4-[2-[[2-hydroxy-2-(pyridin-3-yl)ethyl]amino]ethyl]phenyl]-4-[4-[4-(trifluoromethyl)phenyl]thiazol-2-yl]benzenesulfonamide and
a pharmaceutically acceptable carrier, and optionally a 5-alpha reductase inhibitor, or an NK-1 antagonist or an alpha-1 adrenergic antagonist or an anti-muscarinic agent.

These compounds are discussed and may be prepared as disclosed in U.S. Pat. No. 5,561,142 and U.S. Pat. No. 6,011,048, which are hereby incorporated by reference. A beta 3 agonist of the invention has been studied in postmenopausal women with OAB and has been found to improve micturition frequency, urge episodes and incontinence episodes in that population.

Generally satisfactory results are obtained when the Beta-3 agonist of the present invention are administered at a daily dosage of from about 0.01 milligram to about 100 milligram per kilogram of animal body weight, preferably given in a single dose or in divided doses two to six times a day, or in sustained release form. In the case of a 70 kg adult human, the total daily dose will generally be from about 0.7 milligrams to about 500 milligrams. Doses of 50 mg or 125 mg or 250 mg or 375 mg are often preferred. This dosage regimen may be adjusted to provide the optimal therapeutic response.

Within this aspect is the genus wherein the compositions comprising a beta 3 agonist and a 5-alpha reductase inhibitor.

Within this genus is the sub-genus wherein the 5-alpha reductase inhibitor is selected from finasteride, dutasteride, turosteride and epristeride.

By the term “finasteride” as used here is meant the compound as designated by 4-azaandrost-1-ene-17-carboxamide, N-(1,1-dimethylethyl)-3-oxo-(5α,17β). FDA approved doses for finasteride are 1 mg and 5 mg, once a day.

By the term “dutasteride” as used herein is meant the compound as designated by (5α,17β)-N-{2,5 bis(trifluoromethyl)phenyl}-3-oxo-4-azaandrost-1-ene-17-carboxamide. FDA approved doses for finasteride are 1 mg and 5 mg, once a day. The FDA approved dose for dutasteride is 0.5 mg, once a day. The FDA approved dose for dutasteride is 0.5 mg, once a day.

Within this aspect is the genus wherein the compositions comprise a beta 3 agonist and an alpha-andrenergic receptor antagonist.

Within this genus is the sub-genus wherein the alpha-andrenergic receptor antagonist is selected from amsulosin, terazosin, doxazosin, alfuzosin, indoramin and prazosin.

By the term “amsulosin” (e.g. Flomax or tamsulosin hydrochloride) as used herein is meant the compound designated as (−)-(R)-5-[2-[[2-(O-ethoxyphenoxy)ethyl]amino]propy]-2-methoxybenzenesulfonamide and salts, hydrates and solvates thereof. Amsulosin is disclosed in U.S. Pat. No. 4,703,063 and claimed in U.S. Pat. No. 4,987,152 as being useful in treating lower urinary tract dysfunction. FDA approved doses include 0.4 mg once a day for tamsulosin hydrochloride.

By the term “terazosin” as used herein is meant the compound 1-(4-amino-6,7-dimethoxy-2quinazolinyl)-4-Rtetrahydro-2-furoyl)carbonylbiperazine and salts, hydrates and solvates thereof. Terazosin is disclosed in U.S. Pat. No. 4,251,532. FDA approved doses include 1, 2, 5 and 10 mg once a day for terazosin hydrochloride.

By the term doxazosin as used herein is meant the compound 1-(4-amino-6,7-dimethoxy-2-quinazolinyl)-4-[(2,3-dihydro-1,4-benzodioxin-2-yl)carbonyl]-piperazine and salts, hydrates and solvates thereof. Doxazosin is disclosed in U.S. Pat. No. 4,188,390. FDA approved doses include 1, 2, 4 and 8 mg once a day for doxazosin mesylate.

By the term “alfuzosin” (e.g. Uroxatral) as used herein is meant the compound N-[3-[(4-amino-6,7-dimethoxy-2-quinazolinyl)methylamino]propyl]tetrahydro-2-furancarboxamide and salts, hydrates and solvates thereof. Alfuzosin is disclosed in U.S. Pat. No. 4,315,007. FDA approved doses include 10 mg once a day for alfuzosin hydrochloride.

By the term “indoramin” as used herein is meant the compound N-[[1-[2-(1H-indol-3-yl)ethyl]-4-piperidinyl]benzamine. Indoramin is disclosed in U.S. Pat. No. 3,527,761.

By the term “prazosin” as used herein is meant a compound of the formula 1-(4-amino-6,7-dimethoxy-2-quinazolinyl)-4-(2-furanylcarbonyl)piperazine. and solvates thereof. Prazosin is disclosed in U.S. Pat. No. 3,511,836. FDA approved doses include 1, 2 and 5 mg once a day for prazosin hydrochloride.

Within this aspect is the genus comprising a beta 3 agonist and an NK-1 receptor antagonist.

Within this aspect, is the genus comprising:

(a) an antagonist of the NK-1 receptor selected from:

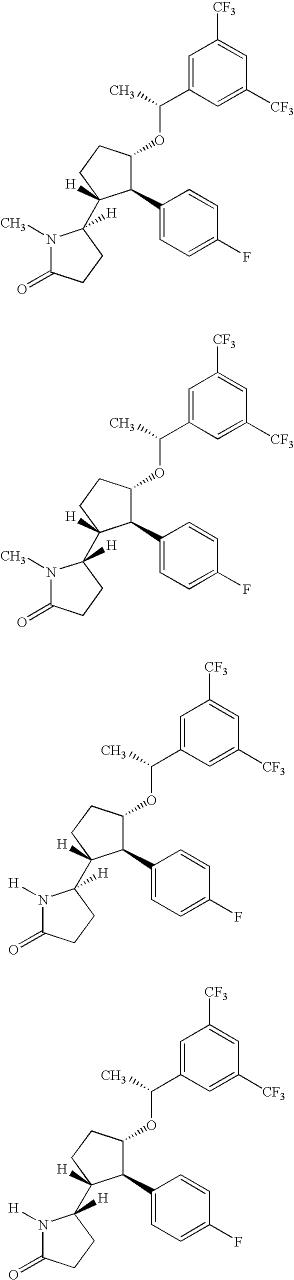

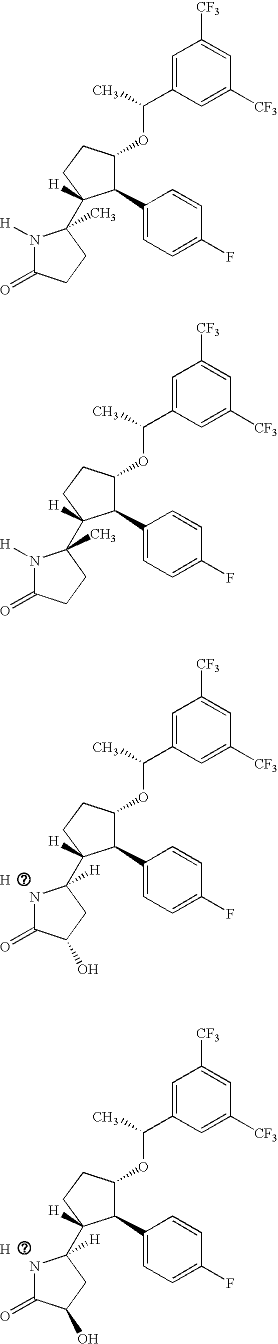

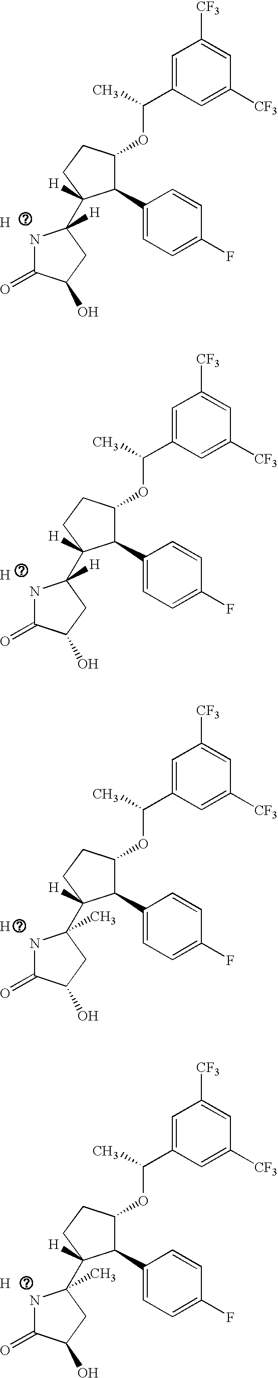

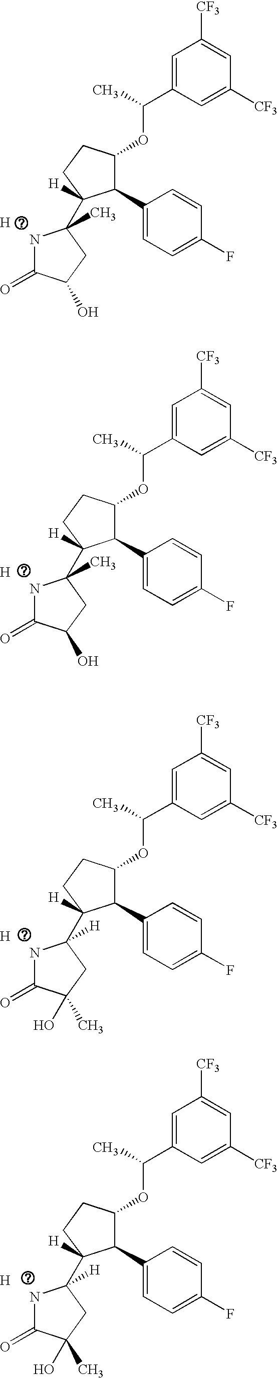

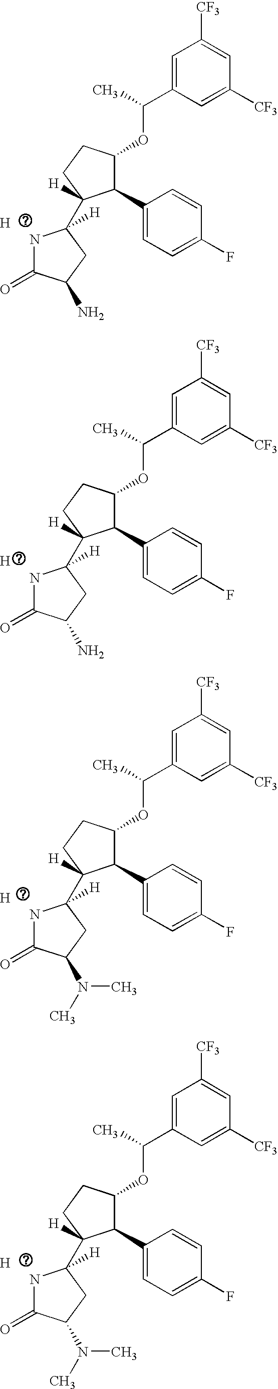

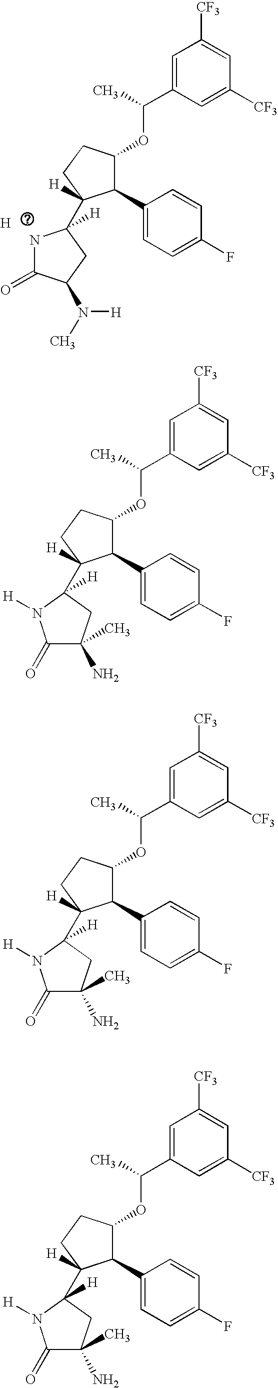

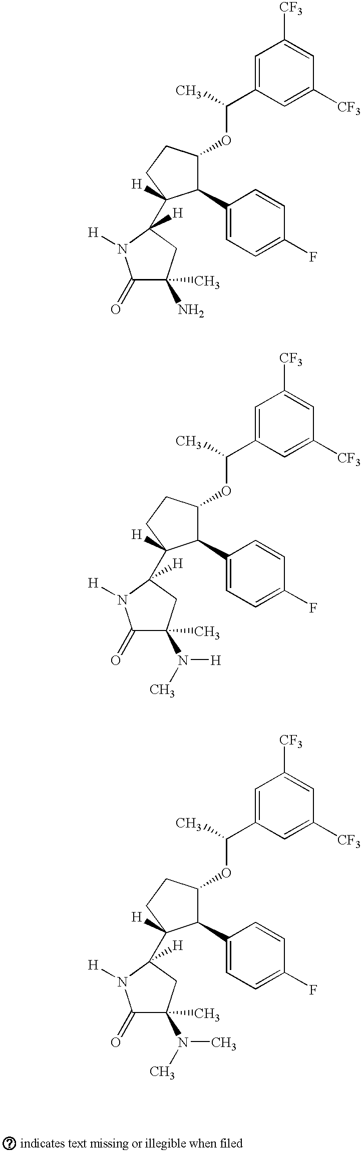

or pharmaceutically acceptable salt thereof;

(b) an antagonist of the NK-1 receptor selected from

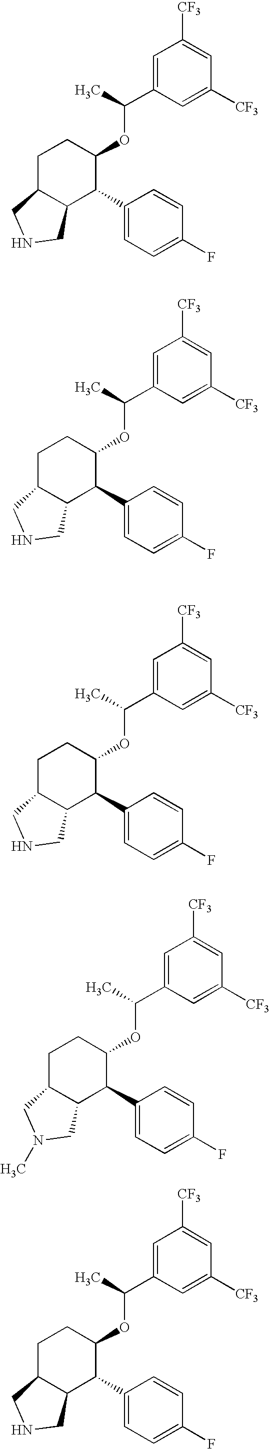

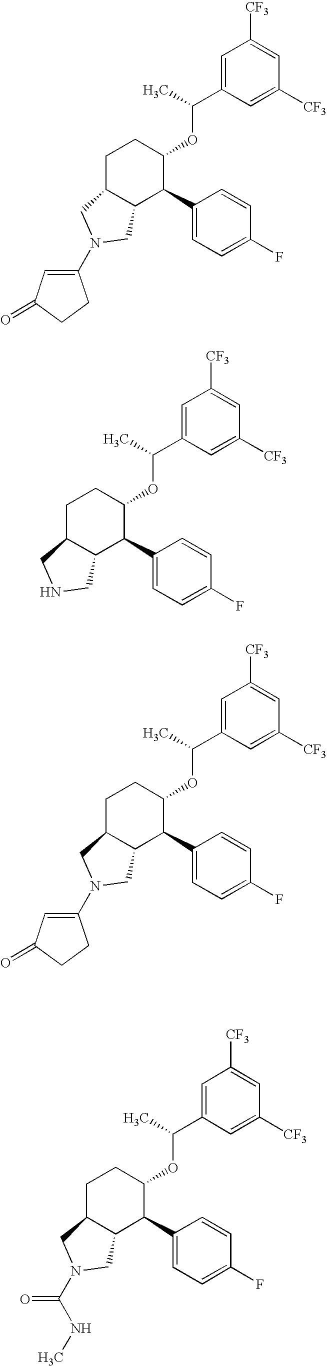

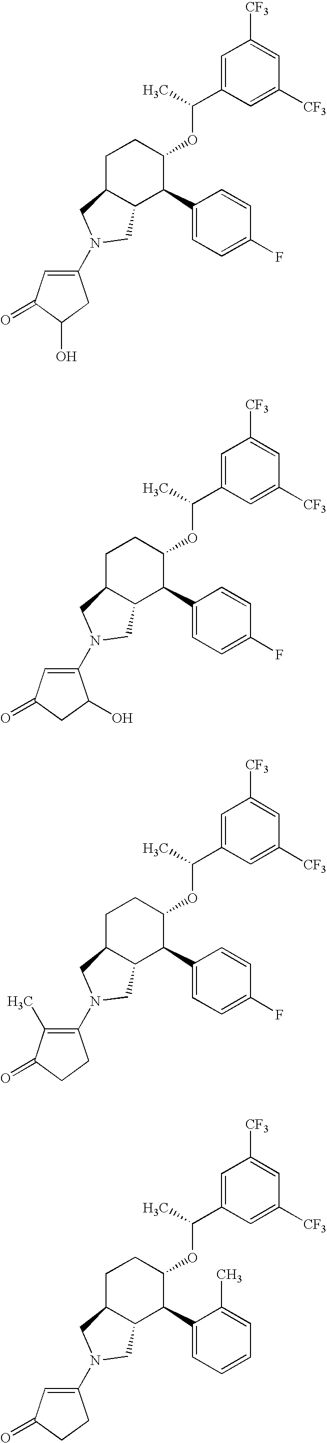

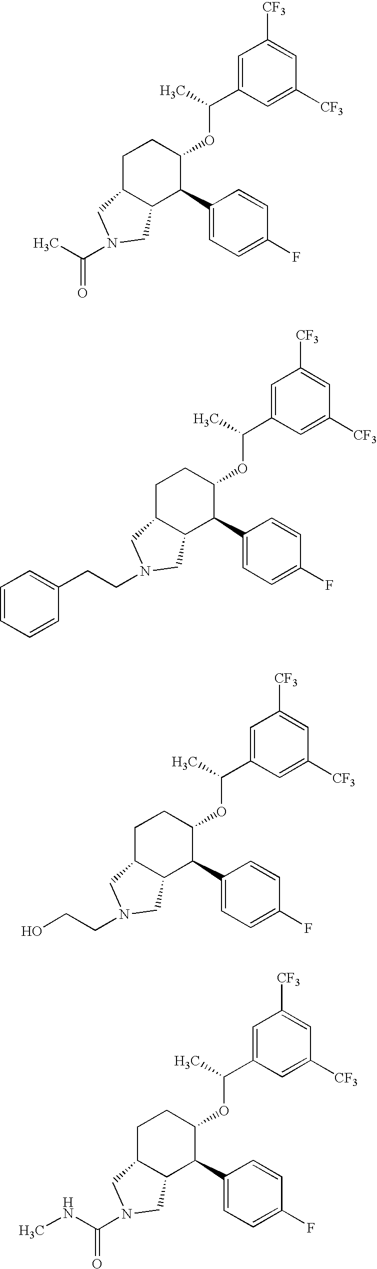

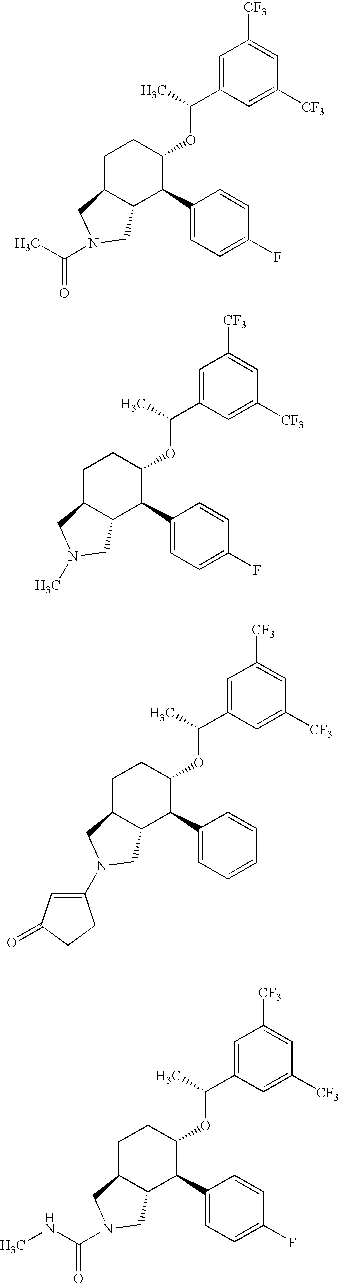

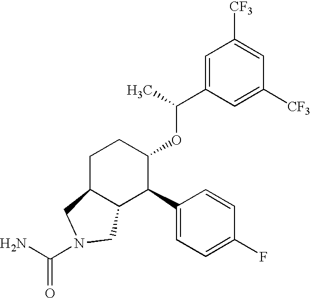

or pharmaceutically acceptable salt thereof;

Within this genus there is a sub-genus wherein the NK-1 receptor antagonists are selected from group (a).

Within this sub-genus is a class wherein there are exactly two active agents: (R)-N-[4-[2-[[2-hydroxy-2-(pyridin-3-yl)ethyl]amino]ethyl]phenyl]-4-[4-(4-trifluoromethylphenypthiazol-2-yl]benzenesulfonamide or a salt thereof, and one NK-1 receptor antagonist selected from group (a).

Within this sub-genus is a class wherein there are exactly two active agents: (R)-N-[4-[2-[[2-Hydroxy-2-(pyridin-3-yl)ethyl]amino]ethyl]phenyl]-4-[4-(4-trifluoromethylphenyl)thiazol-2-yl]benzenesulfonamide or a salt thereof, and the NK-1 receptor antagonist:

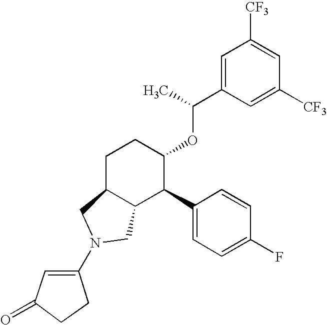

or a pharmaceutically acceptable salt thereof.

Within this genus there is a sub-genus wherein the NK-1 receptor antagonists are selected from group (b).

Within this sub-genus is a class wherein there are exactly two active agents: (R)-N-[4-[2-[[2-Hydroxy-2-(pyridin-3-yl)ethyl]amino]ethyl]phenyl]-4-[4-(4-trifluoromethylphenyl)thiazol-2-yl]benzenesulfonamide or a salt thereof, and one NK-1 receptor antagonist selected from group (b).

Within this sub-genus is a class wherein there are exactly two active agents: (R)-N-[4-[2-[[2-Hydroxy-2-(pyridin-3-yl)ethyl]amino]ethyl]phenyl]-4-[4-(4-trifluoromethylphenyl)thiazol-2-yl]benzenesulfonamide or a salt thereof, and the NK-1 receptor antagonist:

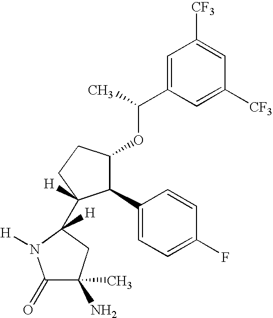

of a pharmaceutically acceptable salt thereof

Within this aspect is the genus wherein the compositions comprising a beta 3 agonist and an anti-muscarinic agent.

For purposes of this specification, anti muscarinc agents included, but are not limited to tolterodine, oxybutynin, trospium, vamicamide, solifenacin, propiverine, S-oxybutynin, temiverine, sanctura, staybla, fesoterodine, SVT40776, 202405 by GlaxoSmithKline, TD6301, RBX9841, DDP200, and PLD179. See, for example, U.S. Pat. No. 5,382,600; U.S. Pat. No. 3,176,019; U.S. Pat. No. 3,480,626; U.S. Pat. No. 4,564,621; U.S. Pat. No. 5,096,890; U.S. Pat. No. 6,017,927; U.S. Pat. No. 6,174,896; U.S. Pat. No. 5,036,098; U.S. Pat. No. 5,932,607; U.S. Pat. No. 6,713,464; U.S. Pat. No. 6,858,650; and DD 106643. See also, U.S. Pat. No. 6,103,747; U.S. Pat. No. 6,630,162; U.S. Pat. No. 6,770,295; U.S. Pat. No. 6,911,217; U.S. Pat. No. 5,164,190; U.S. Pat. No. 5,601,839; U.S. Pat. No. 5,834,010; U.S. Pat. No. 6,743,441; WO2002000652; WO200400414853. These also include trospium chloride, darifenacin and imidafenacin (KRP-197). As will be appreciate by those of skill in the art, these drugs may be administered orally or topically in standard or extended release forms, such as extended release tolterodine, extended relesase oxybutynin and transdermal oxybutynin.

Accordingly, within the aspect of the invention discussed above, there is a genus wherein the anti-muscatinic agent is selected from tolterodine, oxybutynin, trospium, vamicamide, solifenacin, propiverine, S-oxybutynin, temiverine, sanctura, staybla, fesoterodine, SVT40776, 202405 by GlaxoSmithKline, TD6301, RBX9841, DDP200, and PLD179.

Accordingly, within the aspect of the invention discussed above, there is a genus wherein the anti-muscarinic agent is selected from the group consisting of trospium chloride, darifenacin and imidafenacin.

Accordingly, within the aspect of the invention discussed above, there is a genus wherein the anti-muscarinic agent is selected from the group consisting of extended release tolterodine, extended relesase oxybutynin and transdermal oxybutynin.

For purposes of this specification, an effective amount of an anti-muscarinic agent is defined as the dose approved by the FDA at the date this patent application was filed for the class of patient in question. For example, the FDA has currently approved the administration of from 5 mg to 30 mg once a day (in adults) of oxybutynin chloride, extended release, once a day. Similarly, an effective amount of tolterodine tartrate includes 2mg per day of the agent

In another aspect this invention is directed to a method of treating Lower Urinary

Tract Symptoms (LUTS), comprising the administration of an effective amount of (R)-N-[4-[2-[[2-hydroxy-2-(pyridin-3-yl)ethyl]amino]ethyl]phenyl]-4-[4-(4-trifluoromethylphenyl)thiazol-2-yl]benzene sulfonamide or a salt thereof and optionally an effective amount of a 5-alpha reductase inhibitor, or an NK-1 antagonist or an alpha-1 adrenergic antagonist or an antimuscarinic agent.

Within this aspect there is a genus comprising the administration of an effective amount of (R)-N-[4-[2-[[2-hydroxy-2-(pyridin-3-yl)ethyl]amino]ethyl]phenyl]-4-[4-(4-trifluoromethylphenyl)thiazol-2-yl]benzenesulfonamide or a salt thereof and an effective amount of at least one additional active agent selected from group (a) and (b)

Pharmaceutical compositions intended for oral use may be prepared according to any method known in the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. Compositions for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil. Aqueous suspensions contain the active materials in admixture with excipients suitable for the manufacture of aqueous suspensions. Oily suspensions may be formulated by suspending the active ingredient in a suitable oil. Oil-in-water emulsions may also be employed. Dispersible powders and granules suitable for preparation of an aqueous suspension by the addition of water provide the active ingredient in admixture with a dispersing or wetting agent, suspending agent and one or more preservatives.

Pharmaceutical compositions of the present compounds may be in the form of a sterile injectable aqueous or oleagenous suspension. The compounds of the present invention may also be administered in the form of suppositories for rectal administration. For topical use, creams, ointments, jellies, solutions or suspensions, etc., containing the compounds of the present invention may be employed. The compounds of the present invention may also be formulated for administered by inhalation. The compounds of the present invention may also be administered by a transdermal patch by methods known in the art.

The compositions containing compounds of the present invention may be presented in unit dosage form and may be prepared by any of the methods well known in the art of pharmacy. The term “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets or capsules for oral administration, single dose vials for injection, or suppositories for rectal administration. This list of unit dosage forms is not intended to be limiting in any way, but merely to represent typical examples in the pharmacy arts of unit dosage forms. The compositions containing compounds of the present invention may also be presented as a kit, whereby two or more components, which may be active or inactive ingredients, carriers, diluents, and the like, are provided with instructions for preparation of the actual dosage form by the patient or person administering the drug to the patient. Such kits may be provided with all necessary materials and ingredients contained therein, or they may contain instructions for using or making materials or components that must be obtained independently by the patient or person administering the drug to the patient.

By “pharmaceutically acceptable” it is meant the carrier, diluent or excipient must be compatible with the other ingredients of the formulation and not deleterious to the recipient thereof.

The terms “administration of or “administering a” compound should be understood to mean providing a compound of the invention to the individual in need of treatment in a form that can be introduced into that individual's body in a therapeutically useful form and therapeutically effective amount, including, but not limited to: oral dosage forms, such as tablets, capsules, syrups, suspensions, and the like; injectable dosage forms, such as IV, IM, or IP, and the like; transdermal dosage forms, including creams, jellies, powders, or patches; buccal dosage forms; inhalation powders, sprays, suspensions, and the like; and rectal suppositories. The term “therapeutically effective amount” refers to a sufficient quantity of the compounds of the present invention, in a suitable composition, and in a suitable dosage form to treat or prevent the noted disease conditions.

It will be appreciated that when using any combination described herein, both the compound of the present invention and the other active agent(s) will be administered to a patient, within a reasonable period of time. The compounds may be in the same pharmaceutically acceptable carrier and therefore administered simultaneously. They may be in separate pharmaceutical carriers such as conventional oral dosage forms which are taken simultaneously. The term “combination” also refers to the case where the compounds are provided in separate dosage forms and are administered sequentially. Therefore, by way of example, one active component may be administered as a tablet and then; within a reasonable period of time, the second active component may be administered either as an oral dosage form such as a tablet or a fast-dissolving oral dosage form. By a “fast dissolving oral formulation” is meant, an oral delivery form which when placed on the tongue of a patient, dissolves within about 10 seconds. By “reasonable period of time” is meant a time period that is not in excess of about 1 hour. That is, for example, if the first active component is provided as a tablet, then within one hour, the second active component should be administered, either in the same type of dosage form, or another dosage form which provides effective delivery of the medicament.

The compounds of this invention may be administered to patients (humans and animals, including companion animals, such as dogs, cats and horses) in need of such treatment in dosages that will provide optimal pharmaceutical efficacy. It will be appreciated that the dose required for use in any particular application will vary from patient to patient, not only with the particular compound or composition selected, but also with the route of administration, the nature of the condition being treated, the age and condition of the patient, concurrent medication or special diets then being followed by the patient, and other factors which those skilled in the art will recognize, with the appropriate dosage ultimately being at the discretion of the attendant physician.

As discussed above a suitable dosage level of the beta 3 agonist of the present invention, or pharmaceutically acceptable salts thereof, is about 25 to 750 mg per day, which may be given as a single dose or divided into two or three doses per day. Preferably, the dosage range will be about 50.0 mg to 375 mg per patient per day; more preferably about 50.0 to 250 or 100 to 375.0 mg per patient per day. Specific dosages of the compounds of the present invention, or pharmaceutically acceptable salts thereof, for administration include 10 mg, 25 mg, 50 mg, 100 mg, 125 mg, 200 mg, 250 mg, and 375 mg.

A suitable dosage level of the NK-1 receptor antagonist or pharmaceutically acceptable salts thereof, is about 0.001 to 50 mg/kg per day, in particular about 0.01 to about 25 mg/kg, such as from about 0.05 to about 10 mg/kg per day. The dosage range will generally be about 0.5 to 1000 mg per patient per day, which may be administered in single or multiple doses. Preferably, the dosage range will be about 0.5 mg to 500 mg per patient per day; more preferably about 0.5 mg to 200 mg per patient per day; and even more preferably about 1 mg to 10 mg or 5 mg to 50 mg per patient per day. Specific dosages of the compounds of the present invention, or pharmaceutically acceptable salts thereof, for administration include 1 mg, 5 mg, 10 mg, 30 mg, 100 mg, and 500 mg. Pharmaceutical compositions of the present invention may be provided in a formulation comprising about 0.5 mg to 1000 mg active ingredient; more preferably comprising about 0.5 mg to 500 mg active ingredient; or 0.5 mg to 250 mg active ingredient; or 1 mg to 10 or 100 mg active ingredient. Specific pharmaceutical compositions comprise about 1 mg, 5 mg, 10 mg, 30 mg, 100 mg, and 500 mg of active ingredient.

The NK-1 receptor antagonists of group (a) and methods of making same are disclosed in WO2006/00217, published Jan. 5, 2006.

The NK-1 receptor antagonists of group (b) and methods for making same are disclosed in WP2005/073191, published Aug. 11, 2005.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention.

1. A pharmaceutical composition for the treatment of lower urinary tract symptoms (LUTS), comprising a therapeutically effective amount of a beta 3 agonist selected from
N-[4-[2-[[2-hydroxy-2-(pyridin-3-yl)ethyl]amino]ethyl]phenyl]-4-[4-(3-cyclopentylpropyl)-5-tetrazolon-1-yl]benzenesulfonamide; and
2N-[4-[2-[[2-hydroxy-2-(pyridin-3-yl)ethyl]amino]ethyl]phenyl]-4-[4-[4-(trifluoromethyl)phenyl]thiazol-2-yl]benzenesulfonamide or a pharmaceutically acceptable salt thereof and
a pharmaceutically acceptable carrier, and optionally a therapeutically effective amount of a 5-alpha reductase inhibitor, or an NK-1 antagonist or an alpha-1 adrenergic antagonist or an antimuscarinic agent.

2. A pharmaceutical composition according to claim 1 comprising a beta 3 agonist and one additional active agent selected from 5-alpha reductase inhibitor, or an NK-1 antagonist or an alpha-1 adrenergic antagonist or an anti-muscarinic agent.

3. A pharmaceutical composition according to claim 2 comprising a beta 3 agonist and 5-alpha reductase inhibitor wherein the 5-alpha reductase inhibitor is selected from the group consisting of finasteride, dutasteride, turosteride and epristeride.

4. A pharmcceutical composition according to claim 2 wherein the 5-alpha reductase inhibitor is finasteride or dutasteride.

5. A pharmaceutical composition according to claim 2 comprising a beta 3 agonist and an alpha-1 adrenergic antagonist, wherein the alpha-1 adrenergic antagonist is selected from amsulosin, terazosin, doxazosin, alfuzosin, indoramin and prazosin.

6. A pharmaceutical composition according to claim 5 comprising a beta 3 agonist and an alpha-1 adrenergic antagonist, wherein the alpha-1 adrenergic antagonist is selected from amsulosin and alfuzosin.

7. A pharmaceutical composition according to claim 2 comprising a beta 3 agonist and an NK-1 antagonist, wherein the NK-1 antagonist is selected from
(a) an antagonist of the NK-1 receptor selected from: 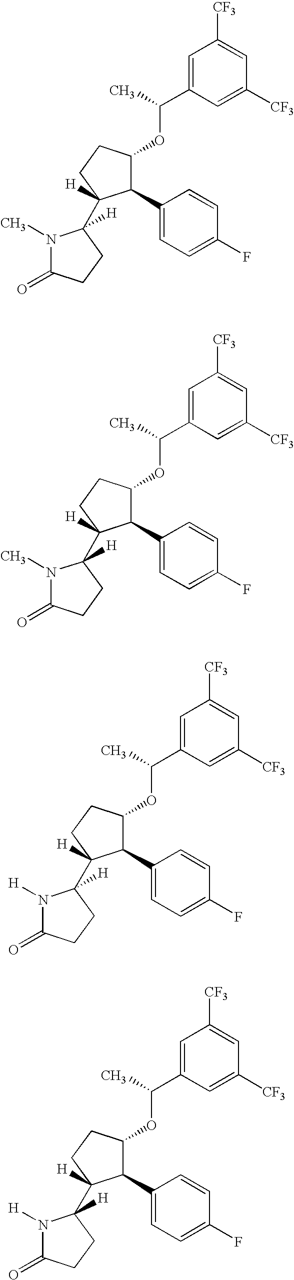 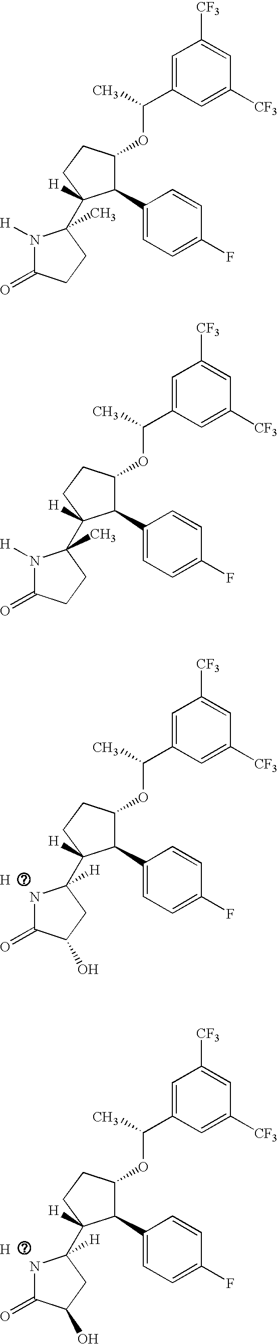 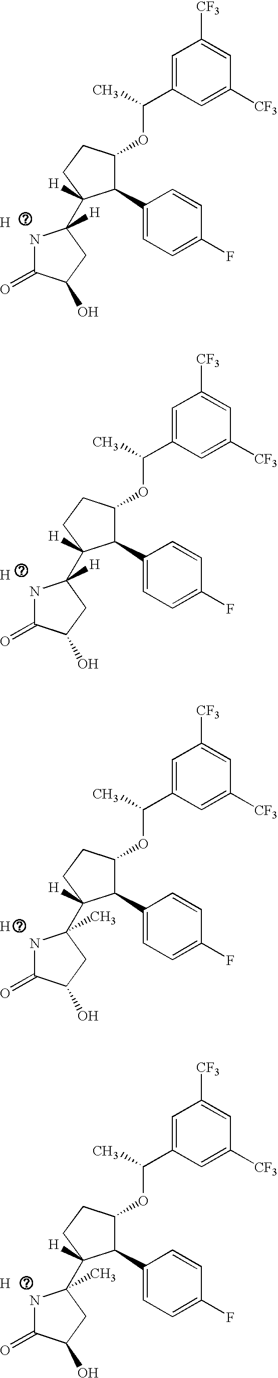 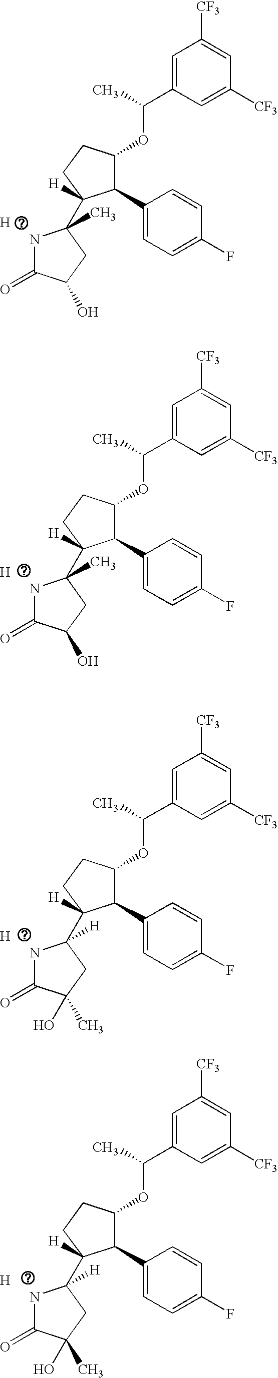 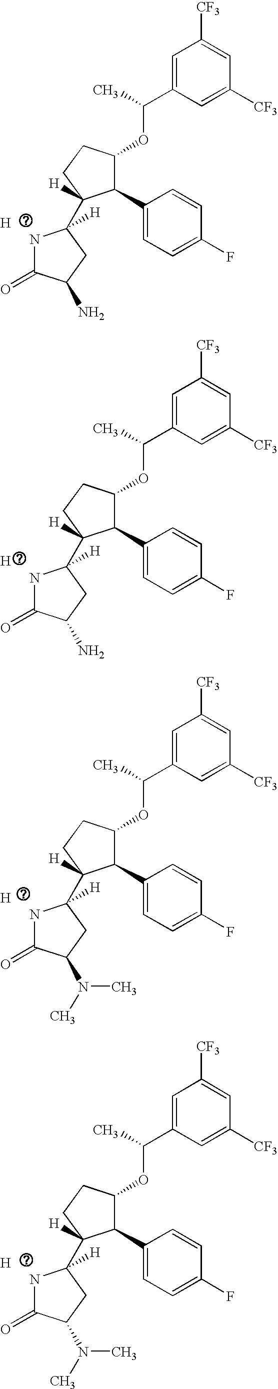 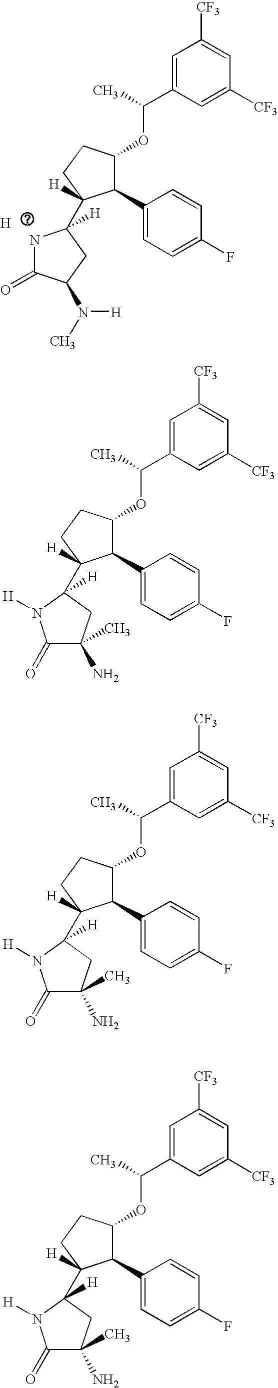 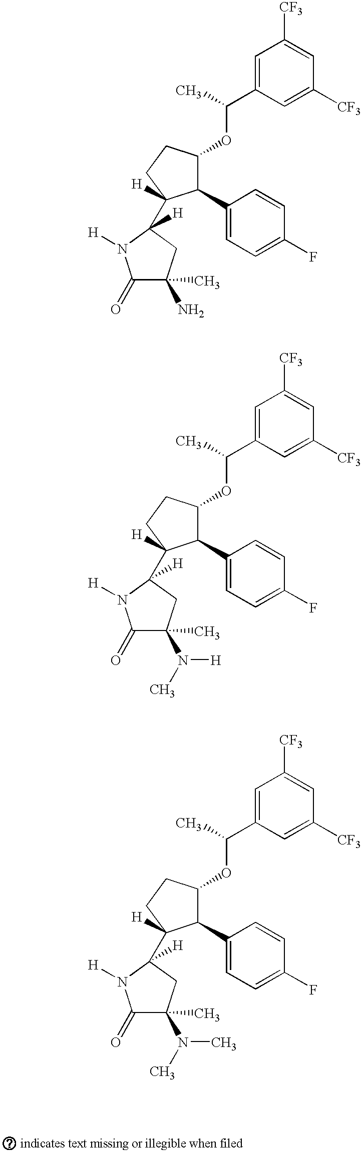
or pharmaceutically acceptable salt thereof;
(b) an antagonist of the NK-1 receptor selected from 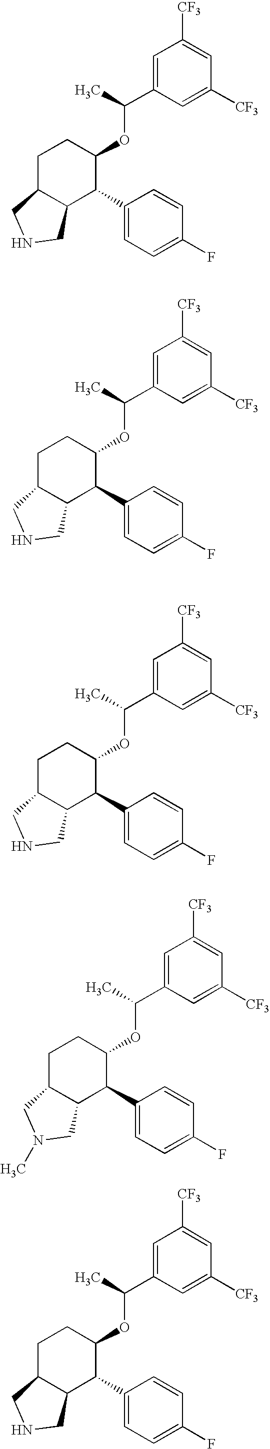 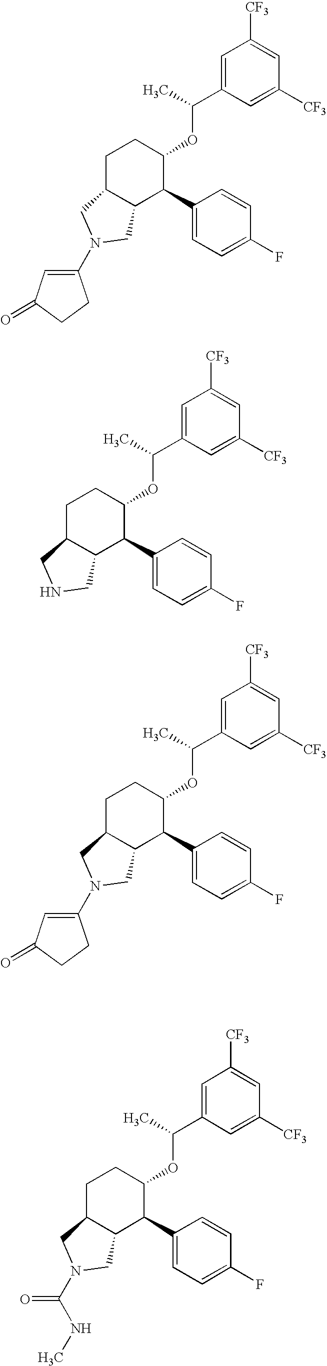 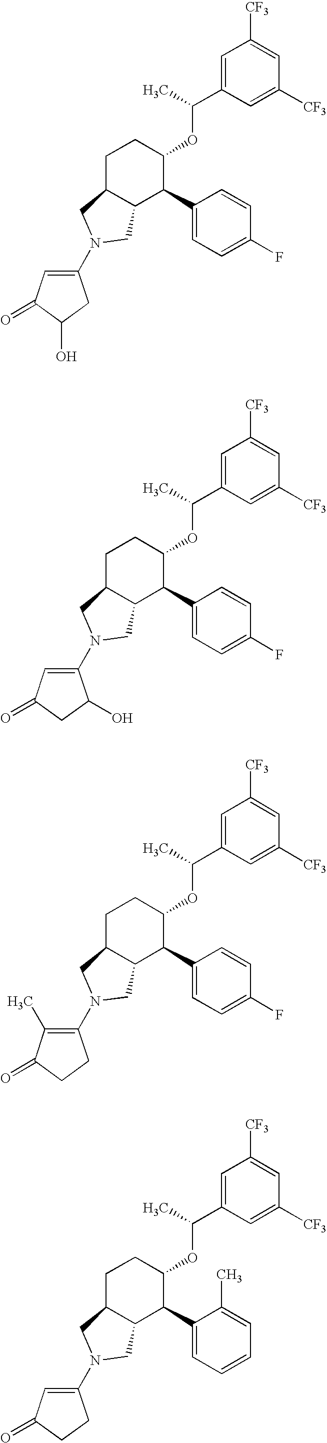 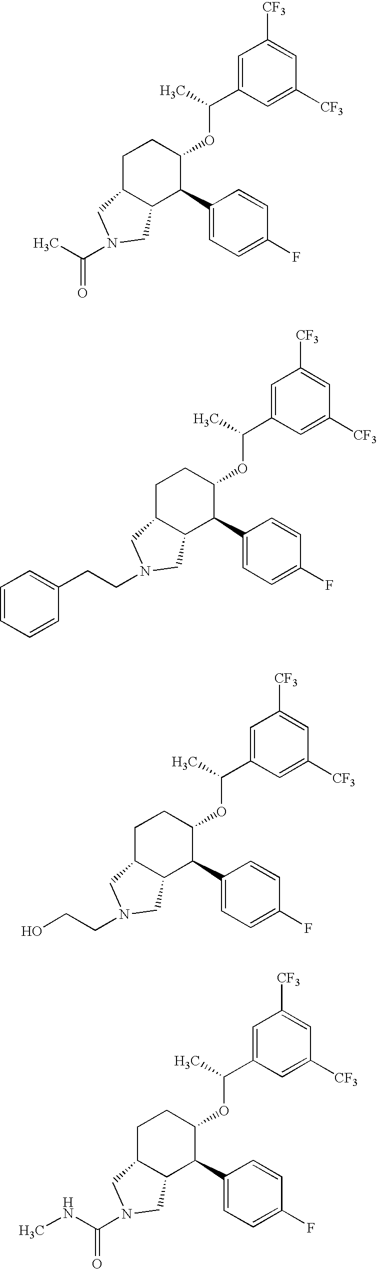 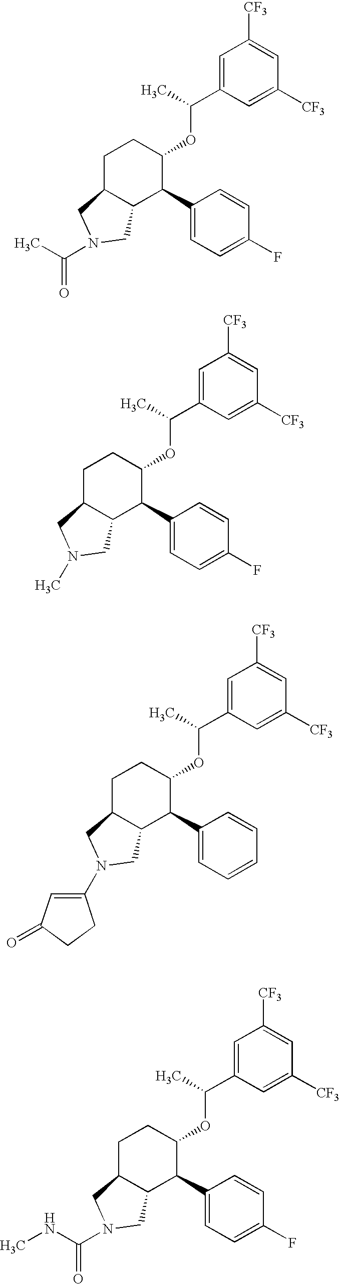 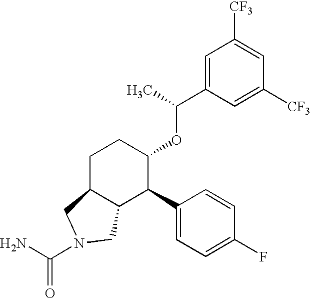
or pharmaceutically acceptable salt thereof

8. A composition according to claim 7 wherein the NK-1 receptor antagonist is selected from group (a).

9. A composition according to claim 7 wherein the NK-1 receptor antagonists are selected from group (b).

10. A pharmaceutical composition according to claim 7 wherein the beta 3 agonist is selected from
N-[4-[2- [[2-hydroxy-2-(pyridin-3-yl)ethyl]amino]ethyl]phenyl]-4-[4-(3-cyclopentylpropyl)-5-tetrazolon-1-yl]benzenesulfonamide; and
2N-[4-[2-[[2-hydroxy-2-(pyridin-3-yl)ethyl]amino]ethyl]phenyl]-4-[4-[4-(trifluoromethyl)phenyl]thiazol-2-yl]benzenesulfonamide, or a pharmaceutically acceptable salt thereof, and the NK-1 receptor antagonist is selected from 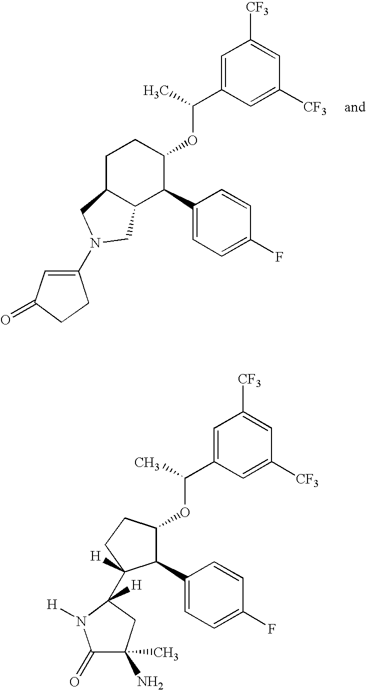
or a pharmaceutically acceptable salt thereof.

11. A pharmaceutical composition according to claim 2 comprising a beta 3 agonist and an anti-muscarinic agent.

12. A pharmaceutical composition according to claim 11 wherein the antimuscarinic agent is selected from tolterodine, oxybutynin, trospium, vamicamide, solifenacin, propiverine, S-oxybutynin, temiverine, sanctura, staybla and fesoterodine.

13. A pharmaceutical composition according to claim 12 wherein the anti-muscarinic agent is selected from tolterodine, and oxybutynin.

14. Use of a composition according to claim 1 for the treatment of Lower urinary Tract Symptoms.

15. A method of treating lower urinary tract symptoms in a patient in need of such treatment comprising the administration of a therapeutically effective amount of a composition according to claim 1.

16. A method of treating lower urinary tract symptoms in a patient in need of such treatment comprising the administration of a therapeutically effective amount of a beta 3 agonist selected from
N-[4-[2-[[2-hydroxy-2-(pyridin-3-yl)ethyl]amino]ethyl]phenyl]-4-[4-(3-cyclopentylpropyl)-5-tetrazolon-1-yl]benzenesulfonamide; and
2N-[4-[2-[[2-hydroxy-2-(pyridin-3-yl)ethyl]amino]ethyl]phenyl]-4-[4-[4-(trifluoromethyl)phenyl]thiazol-2-yl]benzenesulfonamide, or a pharmaceutically acceptable salt thereof and
optionally a therapeutically effective amount of a 5-alpha reductase inhibitor, or an NK-1 antagonist or an alpha-1 adrenergic antagonist or an anti-muscarinic agent.
